(19) United States
(12) Patent Application Publication
(10) Pub. No.: US 2014/0276000 A1
(43) Pub. Date: Sep. 18, 2014

(54) Laser Tracking of Surgical Instruments and Implants

(71) Applicant: Vector Sight Inc., Germantown, TN (US)

(72) Inventors: Michael W. Mullaney, Naples, FL (US); Stephen T. Miller, Scotts Valley, CA (US); William Brian Austin, German Town, TN (US); Todd A. Martens, Denver, CO (US)

(21) Appl. No.: 14/208,742

(22) Filed: Mar. 13, 2014

Related U.S. Application Data
(60) Provisional application No. 61/793,645, filed on Mar. 15, 2013.

(51) Int. Cl. A61B 19/00

(52) U.S. Cl. CPC A61B 19/5244
USPC 600/424

(57) ABSTRACT

A projection system includes a tracking system comprising a first source of light and a first light sensor; a second source of light and a second light sensor, a reflector, and a processing system. The first light sensor may record a first direction the first source is pointed when it receives a reflection of the first light source from the reflector. The second light sensor may record a second direction the second source is pointed when it receives the reflection of the second light source from the reflector. The processing system may determine a point in space corresponding to an intersection of the first and second directions, and may calculate the relationship between the first and second light sources and the reflector.

### PRIORITY

The present disclosure claims priority to and the benefit of the filing date of U.S. Provisional Application 61/793,645, filed Mar. 15, 2013, incorporated herein by reference.

### FIELD OF THE INVENTION

This application relates to systems that aid in locating a particular implant or element of an implant that is disposed within a patient or tracking the position of an instrument relative to a patient or implant when in use.

### BACKGROUND

It is common to use lasers to indicate placement of objects due to the ability of the laser light to illuminate a controlled area of space. For example, in a common household laser level, a laser beam can be modified to emit light in a sector of a plane and then attached to a bubble level such that when the bubble level indicates horizontal, everything illuminated by the laser is at the same height. Another common use of a laser includes generating two laser planes that cross at a common axis of interest. An example of this is in a drill press where two laser planes are both aligned with the axis of the drill so that a user can quickly place the work piece where the hole will be drilled. Both of these examples highlight common uses where the laser system is fixed in a known relationship to the target.

During surgical procedures, the target is not always clearly visible. Often, there is tissue between the surgeon and the target, and the surgeon wishes to disrupt as little of this tissue as possible. A solution described in U.S. Pat. No. 5,782,842 by Kloess et al. incorporates an imaging system for determining an object of interest. The patient is located on a table on an imaging system such as a computed tomography or magnetic resonance imaging system. Once the object of interest is found on the imaging system, the surgeon determines the desired entry point and direction of insertion of the instrument. The relative position of the table and the two laser planes are adjusted to place the intersection of the laser planes along the insertion path. The surgeon then places his instrument at the intersection of these two laser planes, and the lasers visually indicate that the instrument is placed in the proper position.

In U.S. Pat. No. 8,182,149, Heras improves upon Kloess by controlling the position and orientation of the laser planes using a numerical controller. The laser planes are adjustable in at least two degrees of freedom so that the light fan beam can be adjusted to intersect any axis in the workspace. Knowing the position of the laser projectors relative to the imaging system, the desired trajectory of the instrument can be determined on the imaging system and the laser planes can be oriented so that each light fan beam intersects the trajectory. The surgeon can then visually see that the instrument is placed at the intersection of the laser planes. Heras also controls the sweep of the laser projector so that a point along the trajectory can be visualized. This point can be used to visually indicate the proper placement of the instrument along the trajectory. Using this, a surgeon can both align the instrument with the desired trajectory and insert the instrument along the trajectory until it is placed at the desired depth. Heras then describes moving the laser planes to illuminate a new trajectory and depth, allowing the surgeon to insert an instrument along a path that is not just a straight line. This method describes using the imaging system to measure the location and depth of the instrument so that the laser planes can be adjusted at the proper time. One issue that Heras identifies is calibrating the laser plane controller to the workspace and imaging system. The solution Heras uses is placing a calibration device that can be seen on the imaging system into the workspace and adjusting the laser planes to intersect the calibration device properly.

In U.S. patent application Ser. No. 14/044,382, Mullaney describes a similar system where two laser projectors that can generate laser planes are attached to an implant. In this system, the laser planes target a feature of the implant or a feature of the patient that is in a known position relative to the implant. No imaging system is required, as the targeted feature has a known geometric relationship relative to the implant. Therefore, even if the feature is obscured by tissue, the feature can be targeted. An example is targeting of a screw hole on an intramedullary nail. The nail is inserted into a long bone through an entry hole created at one end. The laser projectors are attached to a support structure mounted to the implant at this entry hole. The system can then generate two laser planes to intersect at any trajectory needed to define an axis for placement of an instrument, such as a drill, through the implant feature, such as a screw hole. The patent application also describes a deflection measuring instrument that can determine deformation of the implant, and adjusting the laser planes to match the trajectory needed to properly intersect the deformed implant. Mullaney also describes using the laser modules to illuminate two additional planes of light that intersect, displaying another axis that intersects the first axis. By using these two axes, an instrument can be placed such that certain features are illuminated by these two axes (or all four laser planes), which places the instrument in a desired position and orientation relative to the implant.

The system described by Mullaney improves upon the Heras and Kloess systems not only because it can describe more than a simple axis and depth, but also because it does not require an imaging system, although it may be used with an imaging system. An imaging system such as a CT or MRI is not a regularly available tool in most surgical procedures, is quite expensive, and can be difficult to work around. Other medical imaging systems have other drawbacks. It is advantageous to not require a medical imaging system. However, the system described by Mullaney does require having a known relationship between the features of interest and the laser projection modules. If these features need to be determined during the procedure, for example if the surgeon wants to define the position relative to anatomic landmarks, the position of the anatomic landmarks relative to the laser projection modules has to become known. In addition, minor variations in production processes may mean slight differences in the location of an implant feature.

What is needed is the ability to use the laser projectors to determine the location of the instrument or implant relative to the laser projectors.

### SUMMARY

This application relates to the measurement and display of position information during surgery utilizing laser light sources. Specifically it refers to using a laser source to scan a sector of space where a tool is expected to be located and capture position information using reflections of the laser source on the tool. Further, the laser source can then be directed to project light in defined patterns in the workspace as visual guides for the surgeon.

One embodiment of the present invention relates to targeting of a screw hole in an intramedullary nail. The intramedullary nail is inserted into the canal of a long bone and spans a fracture site. Screws are inserted perpendicular to the long axis of the bone through the bone and the nail. The surgeon must be able to target the screw holes without seeing them, as they are inside the bone. To provide a guide for the surgeon to drill the bone for the screw, a plane of light that intersects the desired drill axis is generated by a laser projector, a second plane of light that also intersects the desired drill axis is generated by a second laser projector, and the surgeon places the drill guide at the intersection of the two planes of light. Since the nail has more than one screw hole, the surgeon has to tell the control system which screw hole he wants to target.

One method for indicating which screw hole the surgeon desires to target is to place a drill guide near the expected entry location. The drill guide includes reflectors which reflect the laser beam. The laser projectors also include a receiver which is a sensor which can be tuned to the same wavelength as the laser. The laser projectors scan the workspace. When the receivers indicate that the drill guide is near one of the screw holes, the laser projectors generate the two planes of light to indicate the trajectory of the closest screw hole.

Although many methods are available for projecting laser light over an area or scanning with laser light, this specific embodiment utilizes projecting light at a moving mirror. By moving the mirror, the direction of the laser can be adjusted. Using technologies such as servo motors or micro-electro-mechanical system (MEMS) chips allows very rapid reorientation of the projected laser. Using a laser diode to generate the laser beam allows the laser beam to be quickly turned off and on. These two devices can be controlled together using a simple numerical controller or a more complicated programmable computer. This embodiment describes the use of a computer processor so the same processor can provide additional functions. Using a moving mirror, a scan pattern can be generated to quickly project the laser beams across a workspace. Reflections from the objects illuminated by the scan will direct light back at the laser projector. Located in the laser projector is a receiver such as a photodiode array.

The drill guide used in this embodiment has spherical features attached to it. These features are highly polished, reflecting the laser without causing much diffusion. The rest of the drill guide has a matte surface so the laser light is diffused and not directly reflected. These features are also located in a specific relationship to each other. The first laser emits light following a specific pattern. As this first laser is directed through the center of the sphere, the laser light is reflected directly back and illuminates the photodiode array, sending a trigger to the computer processor. The computer processor records the direction of the laser based on the position of the mirror at the time of the trigger. This first laser projector continues to scan the workspace and directions of direct reflections are recorded. The second laser conducts the same process. The angular position of the lasers can be converted to lines in a coordinate system of the laser projector. The intersections of the lines will provide the coordinates of the center of the spheres. Knowing the relationship between the spheres and the drill guide, the drill guide location can be determined.

The two scanning processes can be conducted at the same time or at staggered times. If conducted at the same time, the first photodiode array may pick up reflections generated by the second laser projector. This will generate lines in the laser projector coordinate system that likely will not intersect. If the lines don't intersect, they can be ignored. It is also possible to use two different wavelength lasers to scan the workspace so the system can ignore all light signals but the proper corresponding reflection. There may also be additional reflective material in the scan area. This may generate additional points. If the relationship between the spheres on the drill guide is known, any points that don't fit the relationship can be ignored. This should leave just the points that define the location of the drill guide.

Knowing the location of all the screw holes in the nail, the system can determine which screw hole the drill guide is closest to and switch to projecting the two laser planes which define that screw hole axis. Because the laser scanner process is quite fast, the laser can scan a broad area quickly, moving fast enough that the amount of illumination from the laser on any one spot in the scan area is very low. The laser projector can scan for a short period of time, project the laser plane for a longer period of time, switch back to the scanning mode, then switch back to the laser plane projection mode. This can be repeated until the drill guide is moved from the scan area. During this process, if the scan process determines that the drill guide has been moved closer to a different hole in the nail, the projection process can be switched to target the new hole.

The procedure the surgeon can use to select the holes starts with implanting the nail and mounting the laser projector system. The laser projector system starts scanning the workspace. Then the surgeon places the drill guide near the end of the nail where the screw hole of interest is. The system displays one of the holes in that area. The surgeon moves the drill guide toward or away from the laser projector and the other screw holes in the area are displayed. The surgeon then moves the drill guide to the location that displayed the hole the surgeon desires to target. The axis of the hole is illuminated by the two laser planes, and the surgeon aligns the drill guide with the axis of the hole.

A second embodiment of the invention utilizes the same system as the first and targets the screw holes in the same manner. The difference is the location of the screw holes are not known by the processor, and this embodiment determines their location prior to implantation of the intramedullary nail through a calibration step that works in the same manner as tracking of the instrument.

An instrument with reflectors on it is used to identify the holes in the intramedullary nail prior to implanting the nail. This can be the drill guide described in the first embodiment or an instrument specifically for identifying the holes in the nail. The specific instrument may be desired for many reasons, such as being easier to hold, more accurate, or that it can be rigidly mounted to the screw hole.

The nail is mounted to the laser projector system. The surgeon places the tool with the reflectors on it so it is aligned with one of the holes in the nail. The laser scans the workspace, and the position of the hole relative to the laser projection system is determined. The tool is then aligned to the next hole in the nail and the scan is repeated. A record of the trajectory of all screw holes in the coordinate system of the laser projection system is stored. Using this laser system and tool in this manner allows the surgeon to determine the hole location so that the nail geometry does not need to be pre-loaded into the program. If the nail geometry is known and pre-loaded into the program, using this scan process allows a calibration of the system to compensate for variations between different nails and the connection between the nail and the laser projector system. In the embodiment where the nail is known but it is desired to calibrate the nail and laser projector system, it may be sufficient to determine just the position of one of the screw holes, and the remaining screw hole trajectories can be calculated using the known relationship between the screw holes and the calibrated position of the one screw hole. One can see that calibrating more screw holes can provide increased accuracy, but the actual number of holes calibrated will be chosen based on the desired trade-off between accuracy and the time spent performing calibration.

In the Mullaney application previously mentioned, U.S. application Ser. No. 14/044,382, use of a deflection measuring probe is described to compensate for bending of the nail once inserted. Another embodiment of the current invention incorporates the use of the tracking function for calibration of the deflection measuring probe. The embodiment is as described above with the addition of the probe inserted into the nail. During the calibration process, an additional step is made where the nail is intentionally deflected. During the deflection, the scanning process tracks the movement of the drill guide, which is mounted to one of the holes at the end of the nail opposite the laser projector system. Both the location of the screw hole and the amount of deflection of the probe are recorded. A relationship between the amount of deflection measured and the change in screw hole trajectory is determined. The probe and drill guide are removed and the nail is implanted. The probe is reinserted in the nail and the deflection is measured. The trajectory of the screw holes are compensated the amount indicated using the deflection/trajectory relationship. The laser projection system then targets the screw holes based on this compensation in the manner described above, allowing the screw hole to be properly targeted even with a change in implant shape due to insertion. It is obvious to one skilled in the art that this calibration can be performed in conjunction with any deflection measuring device. An advantage of using this tracking system for calibrating the deflection is that a relationship between the deflection measuring device and the actual target trajectory is measured directly for each individual nail.

The embodiments described utilize reflective spheres to reflect the laser light back to the receiver. The location of each reflective sphere can be determined by the laser projector system. As described in Mullaney, it is possible to project sufficient information to fully define the desired position and orientation of a device utilizing two axes. The same is true for tracking of a device, where determining two axes of an instrument will provide both the position of the instrument and the orientation of the instrument. To define two axes, all that is required is to define three points, one point being the origin and the other two points being positioned along each axis. Therefore, one embodiment incorporates three reflective sphere rigidly attached to the instrument. If an additional reflective sphere is added, it would be possible to calculate additional coordinate systems which could be used to increase precision of the calculated position of the instrument. In this same manner, it is possible to use less reflective spheres on an instrument if less information is needed. For example, if the only thing needed to be determined is the trajectory, two spheres are sufficient to fully define the coordinates of the trajectory relative to the laser projector system. Further, if only a point is needed, a single sphere could be used to determine the position of the point. This last example could be used when calibrating the deflection, for example, as the calibration instrument could be mounted a known distance from a known screw hole in the nail and the movement of the single reflective sphere could provide the deflection/trajectory relationship. Also, a single sphere is sufficient for a drill guide when used in the first embodiment, as the system only needs to determine which hole the drill guide is nearest, and not the orientation of the drill guide. For use as a tool to calibrate the trajectories of each hole in a nail, a tool with two balls is preferred so the trajectory of each hole is read directly.

Although reflective spheres are described, other reflectors are contemplated. Use of a reflective sphere may require a scan pattern with a very small step over, increasing the time required to complete a scan. One method to increase the size of the reflector is to use a retroreflector array, such as a corner cube reflector array. A quick scan pattern with a large stopover can be initiated, and then a smaller scan around the location of each retroreflector array can be performed to determine the location of the array with greater precision. A ball with retroreflector coating, similar to the balls used in optical surgical navigation, can be used as the retroreflector array. These have an advantage over a shiny surface in that there will be less incidental reflection scattering around the room. These also have an advantage over a flat array in that their shape is consistent no matter what angle they are viewed from. The center of the array can be quickly determined and this can be used in the calculation of position in exactly the same manner as the reflective spheres previously described. Other shapes of retroreflector arrays are contemplated, such as a cylinder which can define a trajectory in the same manner as two spheres. Further shapes may have specific advantages depending upon the intended use.

Although the retroreflector coated balls are similar to those used in one type of computer assisted surgery (CAS) system, namely optical surgical navigation, the process for determining their location is different. In optical surgical navigation, two cameras image an entire workspace at one time, and each image is processed to determine the center of the spheres. The system works with a light source projecting light in a defined wavelength and the image processing is directed to the light read in that wavelength. Typically, infrared light is projected from a source near the two cameras and each camera reads in the infrared light reflected back. The light source is diffuse and untargeted. The location of the spheres is determined by calculating an infinite line defining the angular position of each sphere relative to each camera and then determining the point in three dimensional space that corresponds to the intersection of each vector. The present invention works differently. The photodiode array only records the presence or absence of reflected light in the proper wavelength. The determination of angular position is based on the direction the laser is pointing at the time the reflection is measured.

Additional advantages of the current invention over existing CAS systems also exist. First of all, the output system providing the feedback to the user includes a projector that places the output in the workspace. Most existing CAS systems require the user to look at a video screen to see the CAS system output. Recent advances in CAS systems include placing video screens in the workspace to try to overcome this problem. However, the user is still looking at a graphical representation of the workspace. The use of the laser projector system allows the user to physically align an instrument with a projected alignment in the actual workspace.

A second advantage is that the current invention can mount the tracking system to the anatomy directly. In existing CAS systems, the tracking system is an independent unit outside the workspace, the anatomy and the instruments are both tracked, and the relationship between the two is calculated. If a user wished to use a laser projection system with an existing CAS system, the laser projection system would need to be tracked as well as the instruments. Also, the laser projection system would need to be calibrated in a manner similar to the manner described by Heras for calibrating the laser projector to the imaging system. Since this device uses the same lasers for both tracking and projecting, the coordinate system calculated by the tracking process is the same coordinate system for the projection process.

The embodiments described above cover a simple case of a nail with cross-locking screws. This is a simple case where only trajectories or points need to be determined. Other uses are contemplated. One example is use of this system to place a knee femoral component. A laser projector system can be mounted to the femur. An instrument such as a joint balancing spacer can be tracked. The tibia and spacer would be articulated about the femur as the knee is flexed. The spacer would be used to determine the relationship between the tibial surface and the femoral surface. Using this relationship, the proper size and location of the femoral component can be determined. The laser system could then project two axes. An instrument could be aligned with these two axes and then mounted to the distal femur, and the bone could be prepared relative to this instrument so that the femoral component fits in the proper position. One skilled in the art can readily contemplate additional uses for the device in other surgical procedures.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is an illustration of an exemplary laser projection system in accordance with one aspect of the present disclosure.

FIG. 2 is an illustration of an exemplary implant usable with the laser projection system of FIG. 1.

FIG. 3 is an illustration of an exemplary optical system forming a part of the laser projection system in accordance with one aspect of the present disclosure.

FIG. 4 is an illustration of an exemplary MEMS mirror forming a part of the laser projection system in accordance with one aspect of the present disclosure.

FIG. 5 is an illustration of an exemplary drill guide tool adapted for tracking with the laser projection system.

FIG. 6 is an example scanning pattern conducted by the laser projection system.

FIG. 7 is an illustration of the laser projection system during scanning whereby the laser beams are being reflected off a tool back to the receiver.

FIG. 8 is an illustration of an exemplary laser projection system projecting plane sections that define a point and an axis of interest.

FIG. 9 is an example of a tool configured so that the laser projector system can determine both position and orientation of the tool.

FIG. 10 is an example of a tool configured to be used as a calibration device.

FIG. 11 is an illustration of an exemplary laser projection system using the scanning process to calibrate both the nail screw hole position and the deflection characteristics of the deformation measuring probe.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For simplicity, in some instances the same reference numbers are used throughout the drawings to refer to the same or like parts.

The exemplary laser projection systems disclosed herein are arranged to direct the placement of an implant, such as bone screws, intramedullary nails, hip stem and cup implants, knee replacement implants, and others. These laser systems both project visual information to guide the surgeon and use the lasers to track items in the work space. Visual information projected may include an axial trajectory identifying the location of screw holes or may include an axial trajectory identifying other features of the implant for anchoring or for general implantation or more generally it could be one or more axial identifiers that correspond to such things as a coordinate system. The tracking process can track the location of a point, the trajectory of an axis, or the position and pose of a coordinate system. The point, axis and coordinate system all are associated with a particular implant or instrument of interest. One system described herein is used to track a drill guide for alignment with a screw hole and to display the desired position of the drill guide to properly target the screw hole in an intramedullary nail in a patient. The system generates a laser marker that shows a surgeon where to drill and at what angle to drill to engage the interlocking screw hole in the intramedullary nail. It should be noted that this is merely a single axis application and its description herein is chosen for the sake of simplicity and no such limitation is anticipated or required. It is further anticipated that this single axis example would be expanded to include a full coordinate system definition through the use of multiple axes each defined in similar ways.

FIG. 1 shows an exemplary laser projection system 100 in accordance with an exemplary aspect of the present disclosure. The system in FIG. 1 is shown connected to an intramedullary nail, referred to herein as implant 102. While shown as a nail, the implant 102 could be any implant where axial targeting may be useful whether it be one, two or more axial trajectories that need be identified. The implant 102 may also be a temporary implant placed in or on the bone as a reference marker so that the relationship between the bone and the laser system 100 can be defined. In the example described, the nail is the implant 102, and the features formed on the implant 102 that are not visible to a surgeon are interlock holes configured to receive an interlock screw. The laser projection system 100 may be used to guide an instrument, such as a surgical drill, and may be used to guide an additional connecting implant, such as an interlock screw, into the screw holes in the nail 102 when the nail is disposed within a patient.

The laser projection system 100 includes a laser projector mount 160, a plurality of laser projectors 162, and a processing system 164. The laser projection system 100 also includes a surgical instrument or tool 166. The tool 166 incorporates reflectors 167.

An exemplary implant that may be used with the laser projection system 100 is the intramedullary nail 102 shown in FIG. 2. The nail implant 102 includes a distal end 104, a proximal end 106, and includes interlock holes 108 arranged to receive the interlocking screws (not shown). In this embodiment, the nail implant 102 also includes an adapter interface 110 at the proximal end 106 shaped and configured to align with and connect to an adapter linked to the laser support system during use.

The processing system 164 is a computer system including a processing unit containing a processor and a memory. An output device, such as a display and input devices, such as keyboards, scanners, and others, are in communication with the processing unit. Additional peripheral devices also may be present. The processing unit and peripheral devices may be mounted on the laser projector mount or located remotely from it. Data may be communicated to the processing system 164 by any known method, including by direct communication, by storing and physically delivering, such as using a removable disc, removable drive, or other removable storage device, over e-mail, or using other known transfer systems over a network, such as a LAN or WAN, including over the internet or otherwise. Any data received at the processing system may be stored in the memory for processing and manipulation by the processor. In some embodiments, the memory is a storage database separate from the processor. Other systems also are contemplated.

The processing system 164 may be configured and arranged to receive information over the wire 140, or through wireless communication methods that represent information or signals from the laser projector 162. One set of information is location data generated during the tracking of the tool, 166. Using this information, the processing system 164 may be configured to calculate and output values or data representing the position of implant features, such as the interlock holes 108 of the nail implant 102, even when the implant 102 is not visible to the surgeon. The system uses these features to identify axes that allow a surgeon to access the implant in the patient in an effective manner. For example, a surgical guide such as a drill guide may be aligned with the screw holes based on settings output from the processing system 164.

Here, the laser projection system 100 includes two laser projectors 162 attached to the nail via the laser projector mount. The two laser projectors are offset a similar distance from a centerline of the nail and offset anterior to the nail.

The laser projectors 162 include an optical system 220 formed therein. This optical system is shown in FIG. 3. A main objective of the optical system 220 is to provide a beam of light that originates from a given point in space that can be commanded to point at an arbitrary point in space. A working envelope 222 identifying the area or region within which a beam can be directed from the laser projectors 162 is shown in FIG. 3. Although it can take many forms, here it is conical in nature. It could also be pyramidal or some other polygonal form.

The optical system includes a laser source 226, a collimator 228, a folding mirror 230, a photo diode array 232, a MEMs mirror 234, and an expansion lens 236. In this embodiment the laser source 226 is a laser diode. Typically, these generate an elliptical conical diverging beam 221 which is passed through the collimator 228 to create a straight or converging beam 229. This beam is then directed to the folding mirror 230. The folding mirror 230 is provided to, among other things, make the optical system more compact. However, this isn't necessary and will depend on the particular packaging requirements. After the folding mirror 230, the beam is directed to a micro-electro-mechanical system (MEMS) two-axis gimbal-less mirror 234 which bounces the light beam off in a desired elevation and rotation relative to the nominal. The MEMS mirror 234 is shown in more detail in FIG. 4. As can be seen, the MEMS mirror 234 includes a base frame 240 and a mirror 242. The MEMs mirror is rotatable about a first axis 244 and a second axis 246. Because some devices such as the MEMS mirror 242 have a limited angulation capability, the expansion lens 236 is used as shown in FIG. 3. In this embodiment, the expansion lens 236 expands the working envelope from roughly+/−2 degrees to +/−22 deg. Although this embodiment utilizes MEMS technology, other more traditional means are available to manipulate a mirror in two-degrees of freedom such a motors, piezoelectric elements etc. Also other light sources other than lasers are envisioned along with alternative means of collimating a light source.

The tool 166 is shown in FIG. 5. In this embodiment, the tool 166 is a drill guide. Incorporated into the tool are one or more reflective features, in this case polished spherical surfaces 167. These are mounted in a known position relative to the tool, in this case a tube 120 for guiding a drill, not shown. The spheres 167 are aligned with the inner diameter of the tube 122 so that the trajectory of the drill is known if the locations of the spheres are known. The tool also includes a handle 124 so that the tool can be held without obscuring the reflectors. Other tools may not require a handle depending upon how they are used.

In order to track a tool, the laser projector 162 sweeps a portion of the working envelope 222 in a defined pattern as shown in FIG. 6. Although any scan pattern may be used, one optional scan pattern 310 is shown in FIG. 6 as a Lissajous curve that has progressive coefficients such that a given area bounded by the rectangle joining points 312, 313, 314, and 315 is essentially painted with the scanning beams. FIG. 6 shows this exemplary pattern 310 as it would appear when striking a flat surface 311 located a distance from the laser projector 162. This pattern 310 is created by aiming the laser beam 200 emitted from the first laser projector 162a. A similar pattern or the same pattern can be swept using the laser beam 201 emitted from the second laser projector 162b.

The purpose of the scan process is to sweep the laser beam over the area until it crosses over the reflector. FIG. 7 shows the laser beam 200 emitted from laser projector 162a striking the reflective sphere 167a. The reflective sphere bounces the laser beam back, shown in the figure as 202. During the scan, laser projector 162b shines the laser beam 201 onto the reflective sphere 167a, which send the reflection 203 back to the laser projector. Since the laser projector 162 also contains a photodiode array 232, any beam that is reflected back to the MEMS mirror 234 directly will trigger an event in the photo diode array 232. This event trigger can then be used to capture the commanded mirror angles at the time the trigger occurred. Knowing these angles and the nominal location and pose of the mirror one can obtain the line of sight for each laser projector 162. Knowing this information from two separate laser projectors at different positions allows one to accurately calculate the position in space of the center of a sphere. The processor 164 records the position of the mirror 234 when the photodiode array 232 receives one of the reflections 202, 203. The scan continues until all laser projectors 162 scan all the reflectors 167. Further, if the tool 166 is in motion, the scan continues, tracking the position of the reflectors 167 in movement. In the exemplary embodiment, each tool 166 has two spheres 167, and knowing the center point of both spheres, the axis of the tool can be determined.

This entire process can also be expanded to discover multiple axes thus providing either an axis with locations annotated or axes that intersect with one another leading to the formation of a complete coordinate system which in totality is the means for placing an object in 3d space. Further, this process can be used to track an instrument, such as the drill guide 166, or implant, such as the nail 102, relative to the laser system 160. Finally, the same lasers that are used to scan the field of view also can be used to draw the target axes. The laser would move through the scan pattern at very high speed such that all points in the field of view would be illuminated the same amount. Additionally, the laser system could display the target points or axes as previously described for a given amount of time, then for a very brief time, run through the tracking process, the processor can update the desired target points or axes, and the laser system can display the new target points or axes. This can be repeated continuously as needed. By timing this process so the laser system displays the target points or axes for a greater length of time than it does scanning the field, the target points or axes will appear brighter than the rest of the scanned area. Because the laser projector can move the laser at very high speeds, the amount of time required to scan the surgical workspace is considered to be short enough that the illumination of the workspace from this process will not interfere with the visualization of the projection of information or any other visualization requirements.

Drawing the target axes for the surgeon to visualize uses a process like the scanning process but in reverse. Referring to FIG. 8, each laser projector 162 can target a specific point 174. Laser projector 162a targets point 174 with laser beam 173. Laser projector 162b targets point 174 with laser beam 172. If they are both targeting the same point in 3D space, the laser beams from each projector will cross at the point in space. Each laser projector 162 can then be redirected to target a second point 175, sweeping along an angle between the two points. If each laser projector cycles between these two points, light will illuminate a section of a first plane 170 and a second plane 171. If the two light sources are not coincident, then two plane sections can be illuminated such that the intersection of the two planes is the axis of interest 178.

Each laser beam will pass through the air and illuminate the objects in their path. Typically, the light will strike the patient or surgical drapes. The user will place the instrument or implant, in this embodiment the drill guide 166, in the area that is illuminated. Each light source will project a curve on the drill guide.

Theoretically, each laser projector 162 defines an infinite plane. Practically, as shown in FIG. 8, each laser projector 162 can illuminate only a sector of a plane 170, 171 within the working envelope of the laser projector. By selecting the same point to define one edge 172, 173 of each illuminated plane sector 170, 171, the illuminated axis of interest includes on it a point of interest 174. This point of interest can be aligned with a feature of the implant or instrument. For example, a drill could be inserted in the drill guide 166 along the illuminated axis of interest 178 until a mark is aligned with the point of interest 174, indicating that the target depth of the drill has been reached.

A different embodiment of the tool is shown in FIG. 9. Tool 136 is a different configuration of drill guide Like the previous embodiment, the drill guide includes a tube 130 and a handle 134. Three reflective spheres 137 are mounted on a frame 132 that is located relative to the tool in a known geometric relationship. Although the embodiment of the tool is a drill guide, a slotted guide could replace the tube 130, creating a saw cutting guide. Other tools attached to the frame 132 are also contemplated. Having three balls mounted to the tool allows the laser projector system to locate both the position and orientation of the tool.

In another embodiment, the tool shown in FIG. 10 is a calibration rod 300 that includes a cylindrical segment 302, a spherical end 304, and a mounting end 306. A retroreflective surface coating on the cylindrical shaft 302 and the spherical surface 304 ensures that a signal is returned. Therefore, the cylindrical shaft 302 and the spherical surface 304 of the calibration device 300 will provide an endpoint and an axis.

The reflection from the cylindrical axis of the calibration rod 300 is imaged with the photo diode array 232. For the axis of a cylinder, knowing two lines of sight from a given point allows the formation of a plane. Having a second point from which two lines of sight are known creates a second plane, the intersection of these planes is the axis. If the distance from the endpoint of the calibration device 300 to the implant 102 is known and the endpoint and axis of the calibration rod can be discovered then the location and axis orientation of the interlock hole that the calibration device 300 is placed in is also known. All screw locations for a given nail can be calibrated using this method. In most instances, since the screw hole locations are known relative to each other with a fair bit of precision, the targeting of a single screw hole may suffice.

An additional embodiment incorporates the laser projector system with a deflection measuring device. Any deflection measuring device could be incorporated into the system. In the recent U.S. patent application Ser. No. 13/868,759, filed Apr. 23, 2013, titled Measurement and Resulting Compensation of Intramedullary Nail Deformation (Mullaney, et al.) a method of measuring the absolute deflection of the nail once the nail was implanted through the utilization of a deflection probe inserted into the nail is described. Utilizing a device similar to this in conjunction with the laser projection system allows for calibration of both the screw hole position relative to the laser projection system as well as the change in position of the screw hole relative to the amount of deformation measured in the probe.

FIG. 11 shows a device that calibrates the deformation measuring probe. The laser projector system 100 is mounted to the nail 102 at the adapter interface 110. The calibration device 300 is mounted to a screw hole 108 at the opposite end 104 of the nail 102. The deformation probe 120 is inserted into the nail 102. The laser projector system scans the workspace using a scan pattern 310 and determines the position of the calibration device 300. This establishes the deformation measured by the probe of the nail in the undeformed state as well as the location of the screw hole in the nail in the undeformed state. If the deformation characteristics of the nail relative to the probe were known, this is sufficient to then determine the change in trajectory of the screw hole based on the amount of deformation measured. The user can continue to scan the mounted calibration device 300 while deflecting the nail 102 and measuring the deflection of the nail using the deformation probe 120. Recording the positional data of the calibration device relative to the deflection data of the deformation probe, a relationship between the trajectory of the screw hole and the amount of deformation measured by the probe can be established. Therefore, this device can calibrate both the position of the nail relative to the laser projection system and the relationship between the deformation measuring probe value and the actual change in shape of the nail.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

1. A projection system comprising:
a tracking system comprising:
a first source of light and a first light sensor;
a second source of light and a second light sensor, and
a reflector,
wherein the first light sensor records a first direction the first source is pointed when it receives a reflection of the first light source from the reflector,
wherein the second light sensor records a second direction the second source is pointed when it receives the reflection of the second light source from the reflector;
a processing system configured to determine a point in space corresponding to an intersection of the first and second directions, and to calculate the relationship between the first and second light sources and the reflector.

2. The projection system of claim 1, wherein the first and second sources of light are lasers.

3. The projection system of claim 1, wherein the first and second light sensors are photo diodes.

4. The projection system of claim 1, wherein the reflector is connected to an implant.

5. The projection system of claim 1, comprising an implant and a tool, wherein the tracking system is connected to the implant, and wherein the reflector is connected to the tool.

6. The projection system of claim 5, wherein the tool is structurally configured to aid with calibrating the connection between the tracking system and the implant.

7. The projection system of claim 1, wherein the first and second sources of light are configured to also display information that assists the surgeon in placing tools in surgery.

8. A laser projection system comprising a tracking system, the tracking system comprising:
one or more reflectors;
a first laser source having multiple degrees of freedom such that any point in space within a field of view can be targeted at a rapid rate;
a first receiving device that is triggered when illuminated by light from the first laser source that has reflected back from the one or more reflectors;
a second laser source having multiple degrees of freedom such that any point in space within the field of view can be targeted at a rapid rate; and
a second receiving device that is triggered when illuminated by light from the second laser source that has reflected back from the one or more reflectors; and
a processing system configured to determine the position of the one or more reflectors relative to the first and second laser sources.

9. The projection system of claim 8 wherein the reflector is a reflective sphere.

10. The projection system of claim 8 wherein the one or more reflectors are an array of reflective objects distinctly identifiable.

11. The projection system of claim 10 wherein the array of reflective objects are spheres.

12. The projection system of claim 8 wherein the one or more reflectors are shapes coated with retroreflective coating.

13. The projection system of claim 8 further comprising an implant, and wherein the tracking system is attached to the implant, and wherein the one or more reflectors is respectively associated with one or more tools, the tracking system is configured to determine the position of the one or more tools relative to the implant or patient.

14. The projection system of claim 13 where the first and second laser sources are also configured to project forms onto the patient, implant and or instrument for the purposes of proper placement.

15. The projection system of claim 13 further comprising a tool associated with the one or more reflectors, wherein the tool is configured to calibrate the relationship between the laser tracking system and the attached implant.

16. A method of determining positional information for use during surgery comprising:
providing a tool with at least one reflector and a laser projector system with at least a first laser proctor and a second laser projector, each being associated with a respective first light sensor and a second light sensor;
scanning a workspace with a first laser beam of the first laser projector;
capturing a first reflection of the first laser beam from the at least one reflector with the first light sensor located near the first laser projector;
recording the orientation of the first laser beam when the first reflection is captured;
scanning the workspace with a second laser beam of the second laser projector;
capturing a second reflection of the second laser beam from the at least one reflector with the second light sensor located near the second laser projector;
recording the orientation of the second laser beam when the second reflection is captured; and
calculating the position of the at least one reflector relative to the laser projector system based upon the orientation of the first and second laser beams.

17. The method of claim 16 comprising:
using the position of the at least one reflector to determine what data to present to the user, then
using the first and second laser projectors to project that data into the workspace.

18. The method of claim 17 comprising switching back and forth between scanning the workspace for reflectors and projecting data into the workspace to update and modify the projected data based upon changes in position of the at least one reflector.

19. The method of claim 16 comprising calculating the position of a second reflector of the at least one reflector and using the position of the second reflector to calculate an axis joining the two reflector positions of the reflector and the second reflector.

20. The method of claim 19 comprising directing the projector system to project the location of a projected axis based on the location of the calculated axis by:
calculating the desired start and end point of the projected axis;
directing the first laser projector to emit laser light while sweeping the laser beam repeatedly between the start point and the end point of the projected axis, illuminating a sector of a plane;
directing the second laser projector to emit laser light while sweeping the laser beam repeatedly between the start point and the end point of the projected axis, illuminating a sector of a plane; and
using the two illuminated planes to align an object along the calculated axis.

21. The method of claim 16 comprising calculating the position of a second and a third reflector of the at least one reflectors on the tool and using the second and third reflector to calculate the position and orientation of the tool.

22. The method of claim 16 comprising mounting the tool to an implant, attaching a deformation measuring device to the implant, recording the location of the tool and the corresponding deformation measurement of the deformation measuring device, and calculating a relationship between the tool location and the deformation reading.

23. The method of claim 22 comprising intentionally deflecting the implant while recording tool location and corresponding deformation measurements, and calculating a relationship between the tool location and the deformation reading for a range of implant deflections.

24. The method of claim 23 comprising using the deformation measurement of the implant after implantation along with the relationship between tool location and deformation reading to calculate the deflection of the implant; and
projecting information to properly target a feature of the implant based upon the calculated deflection of the implant.
